(19) United States
(12) Patent Application Publication
(10) Pub. No.: US 2003/0216710 A1
(43) Pub. Date: Nov. 20, 2003

(54) Catheter

(76) Inventor: Robert F. Hurt, Boulder Junction, WI (US)

Correspondence Address: MCDONOUGH, HOLLAND & ALLEN, 555 CAPITAL MALL, 9TH FLOOR, SACRAMENTO, CA, 95814, US

(21) Appl. No.: 10/400,363

(22) Filed: Mar. 26, 2003

Related U.S. Application Data
(60) Provisional application No. 60/367,565, filed on Mar. 26, 2002.

(51) Int. Cl. A61M 25/00

(52) U.S. Cl. 604/523; 604/537

(57) ABSTRACT

An improved drainage catheter having one or more inlet holes along the length of the catheter is described whereby the cross-sectional areas of the successive inlet holes decreases, the decrease first occurring at the inlet hole immediately following the most proximal inlet hole. This change in cross-sectional area alters the typical inflow of fluid into the catheter such that a disproportionately high volume of fluid no longer enters the most proximal inlet hole. This decrease in inflow at the most proximal inlet hole results in less deposition of debris within the catheter at that position. With less deposition of debris at this location, the likelihood of complete catheter failure is reduced. A preferred embodiment is described wherein the change in cross-sectional area results in approximately uniform inflow into all inlet holes.

### CROSS REFERENCE TO RELATED APPLICATIONS

This application claims priority from provisional application 60/0367565 filed on Mar. 26, 2003 which is incorporated in its entirety herein.

### BACKROUND OF THE INVENTION

1. Field of the Invention

The present invention is a medical device, more particularly, a drainage catheter.

2. Discussion of the Prior Art

Catheters of various types are used to drain fluid from a number of different areas within the bodies of animals. In some medical conditions, the flow rate of the fluid to be drained is quite low. As a result, the fluid inflow into the various inlet holes of catheters used to treat these conditions is also quite low.

For example, one application of drainage catheters is to treat hydrocephalus, a medical condition that occurs when cerebrospinal fluid (“CSF”) builds up in the ventricles of the human brain. This build-up causes an abnormal and dangerous increase in intracranial pressure. CSF is formed by the choroid plexuses of the brain and has a normal flow rate between 20 and 30 ml/hr. Drainage catheters are inserted into the ventricles of the brains of hydrocephalic patients to divert their excess CSF. Catheters inserted for such a purpose are termed “proximal catheters.” Some proximal catheters have one inlet hole by which CSF enters. Others have inlet holes along their longitudinal axis that vary in number, shape, distribution, and entrance conditions. The drainage section of these catheters is termed the “proximal end” and the end opposite the drainage section is the “distal end.”

It is commonly thought that only 1 or 2 inlet holes are required to permit adequate flow through proximal catheters and that most inlet holes are redundant. However, when the most proximal inlet hole is obstructed or when that inlet hole permits debris to enter and block the catheter passageway, then the entire catheter fails because no CSF is able to drain from the catheter. It has been widely published that existing proximal catheters have a 30-40% chance of requiring emergency repair in the first year, and an 80% chance of failure after twelve years of implantation. The primary cause of the mechanical failures for these catheters is blockage of the most proximal inlet holes. Blockage is typically caused by CSF debris such as blood clots, cell clusters, brain parenchyma, and choroid plexus and ependymal tissue.

A study of proximal catheters was performed using the analytical tool of computational fluid dynamics (“CFD”). The purpose of the study was to determine the dynamics of inflow into the inlet holes of those catheters. The results of the study demonstrated that 70% of the inflow into catheters having inlet holes of equal area occurred in the most proximal inlet holes. FIG. 5 shows the inflow distribution into a typical proximal catheter having sixteen inlet holes of equal cross-sectional area at eight inflow positions. An “inflow position” occurs at any position along the longitudinal axis of the catheter where at least one inlet hole is located. Inlet hole numbers 1 and 2 shown in FIG. 5 are located at the most proximal inflow position, i.e., the drainage end of the catheter.

As shown in FIG. 5, at low inflow rates fluid inflow into the various inlet holes of drainage catheters is not uniform. This disproportionate inflow causes a disproportionate amount of debris to be deposited within these inlet holes as well as in the catheter passageway at the location of these inlet holes. Because these most proximal inlet holes are located at the drainage end of the catheter, blockage at this point results in drainage failure for the entire catheter.

Numerous designs have been attempted to guard against debris being deposited onto and into drainage catheters. Some attempts have been made to add physical guards to the external surface of drainage catheters. Other attempts have focused on the valves that are used to regulate the fluid flow out of the catheters. The present invention, however, focuses on the catheter inlet holes themselves and the fluid dynamics that underlie the mechanics behind fluid entry into those inlet holes.

### SUMMARY OF THE INVENTION

The present invention is an improved drainage catheter that, at low fluid inflow rates, encourages more fluid than is typical to enter the catheter's distal inlet holes. The present invention accomplishes this by progressively decreasing the cross-sectional areas of the inlet holes as the proximal end of the catheter is approached. This design permits the fluid to flow less preferentially into the proximal inlet holes and more preferentially into the distal inlet holes than the fluid does in currently existing catheters. FIG. 6 graphically illustrates the inflow distribution into one embodiment of the present invention.

As a consequence of the present invention's readjustment of fluid inflow, the deposit of debris is also altered. The debris deposits in the present invention are more likely to be distributed throughout the series of inlet holes rather than concentrated at the most proximal inlet holes. Thus, the relative increase of fluid inflow into the more distal inlet holes decreases the likelihood that the proximal inlet holes, and the catheter passageway at the location of these holes, will become blocked by debris. Though the distal inlet holes in the present invention will likely accumulate more debris than do distal inlet holes in currently existing catheters, any such increase is functionally irrelevant. It is irrelevant because blockage at the distal inlet holes, or in the catheter passageway at the location of those holes, does not impede fluid from draining from the proximal end, i.e., the drainage end, of the catheter. The present invention, therefore, can be maintained in vivo for a longer period of time before the catheter fails, if at all, due to blockage.

### BRIEF DESCRIPTION OF THE DRAWINGS

FIG. 1 illustrates an embodiment of the present invention having two inlet holes at eight inflow positions.

FIG. 2 is a cross-sectional view of FIG. 1.

FIG. 3 illustrates a portion of another embodiment of the present invention having one inlet hole at three inflow positions.

FIG. 4 illustrates a portion of a further embodiment of the present invention having three inlet holes at two inflow positions.

FIG. 5 graphically illustrates the fluid inflow distribution of a catheter having two inlet holes at eight inflow positions wherein all inlet holes have the same cross-sectional area.

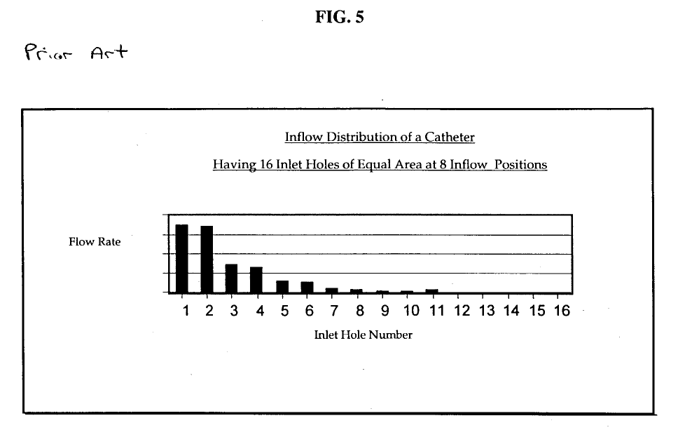

FIG. 6 graphically illustrates the fluid inflow distribution of one embodiment of the present invention having two inlet holes at eight inflow positions wherein the progressive decrease in the cross-sectional areas of the inlet holes was calculated using the curve illustrated in FIG. 7.

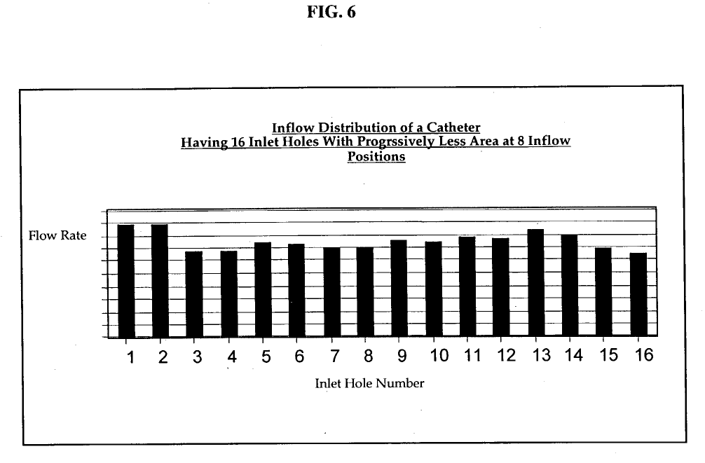

FIG. 7 illustrates the curve that can be used to design one embodiment of-the present invention whereby the fluid inflow distribution is essentially uniform at all inflow positions.

FIG. 8 provides in tabular form the measurements illustrated by the curve in FIG. 7.

### DETAILED DESCRIPTION OF THE INVENTION

The drawings are understood to be illustrative of the concepts disclosed herein to facilitate an understanding of the invention. Further, the drawings are not to scale, and the scope of the invention is not to be limited to the particular embodiments shown and described herein.

Drainage catheters can be improved by designs that force the fluid to be drained into a greater number of inlet holes. The present invention accomplishes this by progressively decreasing the cross-sectional areas of the inlet holes as the proximal end of the catheter is approached.

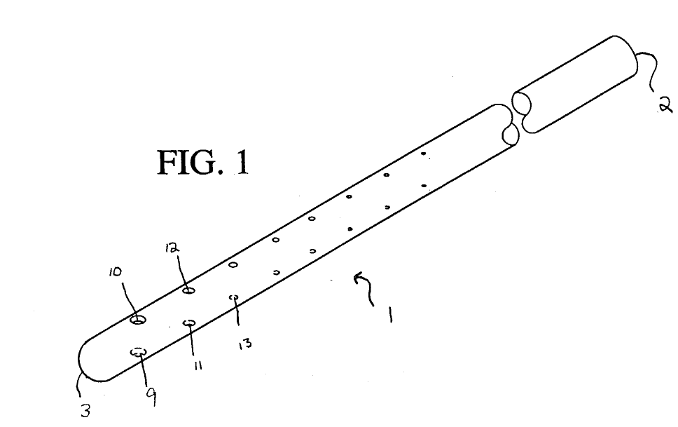

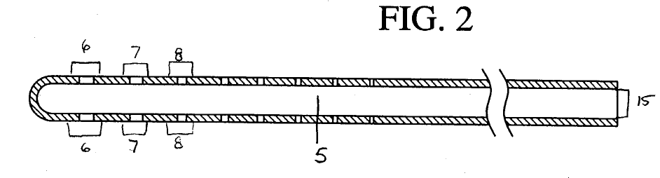

FIGS. 1-4 show a catheter 1 as an elongated tube in accordance with the present invention. The catheter 1 has a proximal end 2 and a distal end 3. The distal end 3 is adapted for implantation into a body cavity of an animal and the proximal end 2 is adapted for connection to means to divert fluid from that particular body cavity. The catheter 1 has an annular wall 4 that defines a central passageway 5. Along the longitudinal axis of the wall 4 two or more inflow positions 6, 7, 8 can be identified. At each inflow position 6, 7, 8 there are one or more inlet holes 9, 10, 11, 12, 13. The inlet holes 9, 11, 13 at each inflow position 6, 7, 8 progressively decrease in cross-sectional area as the inflow positions 6, 7, 8 approach the proximal end 2 of the catheter 1. The catheter 1 so designed may be used to divert fluid from any body cavity where the fluid flow dynamics can be described in the art as “laminar flow” and, more specifically, by mathematically expressing the flow as a Reynolds number between 20 to 800. It is not a limitation of this invention that the inflow positions 6, 7, 8 be equidistant. The space located at the distal end 14 of the catheter 1 functions to maintain the structural integrity of the catheter and may have any length that provides that integrity in order to accomplish the purpose for which the particular catheter is used. It is understood that the overall dimensions of the present invention can vary.

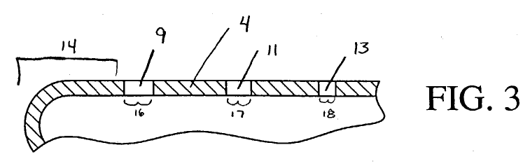

FIG. 3 illustrates one embodiment of the invention. One inlet hole is located at each of three inflow positions along the longitudinal axis of the catheter 1. Each inlet hole 9, 11, 13 has a smaller cross-sectional area 16, 17, 18 than the one preceding it.

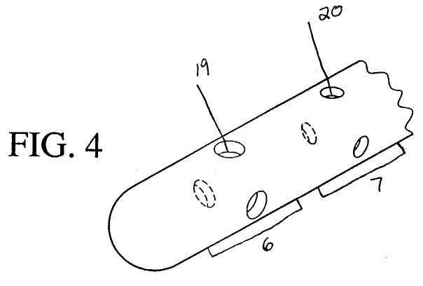

FIG. 4 illustrates another embodiment of the invention. Three round inlet holes are located at each inflow position 6 and 7. The cross-sectional area of each inlet hole at inflow position 7 is less than the cross-sectional area of each inlet hole at inflow position 6.

In an exemplary embodiment, the catheter 1 can be used to divert CSF from the ventricles of a human brain. In this embodiment, the catheter 1 has a length that ranges from about 10 centimeters to about 50 centimeters. The inner diameter 15 of the catheter ranges from about 1.0 millimeters to about 3.0 millimeters. The progressive decrease in the inlet holes 9, 11, 13 cross-sectional areas 16, 17, 18 need not be uniform. However, a method is herein described that results in near equal fluid inflow into the inlet holes 9, 10, 11, 12, 13 at each inflow position 6, 7, 8.

The primary variable that controls fluid inflow into a proximal catheter is the distribution of the total hole areas along its longitudinal axis. “Total hole area” is defined as the sum of all the inlet hole areas at a given inflow position 9, 10. “Inlet hole area” is defined as, and is used interchangeably with, the cross-sectional area of one inlet hole. The phrase “distribution of the total hole areas” is understood to mean the pattern of change in the total hole areas along the longitudinal axis of the catheter 1. Through the tools of computational fluid dynamics and experiment, the distribution of the total hole areas was calculated and optimized to approximate equal inflow into each inflow position for a number of catheter operating conditions, typical implant positions, and body locations. This optimization was accomplished by numerically solving the conservation equations involving mass, energy, and momentum that govern the flow fields to and within the subject catheters. Total hole areas were adjusted for each computational trial until approximately equal inflows were obtained at each inflow position for every catheter analyzed in the study.

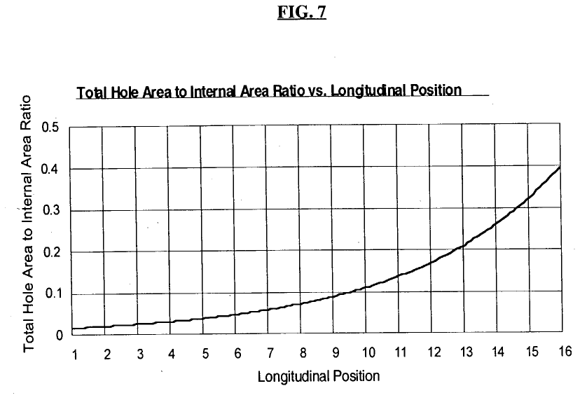

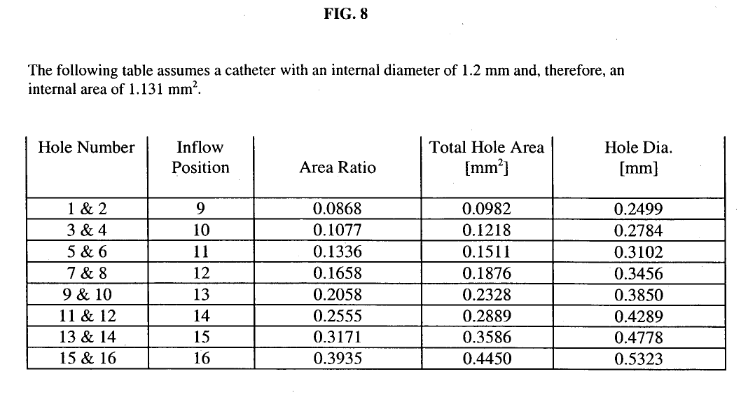

FIG. 7 displays a total hole area distribution curve that provides the means for producing approximately equal inflows in proximal catheters having four to sixteen inflow positions. This distribution curve was generated by compiling all the calculations from the CFD analysis and is, therefore, a generalized curve that can be applied to the manufacture of proximal catheters irrespective of any particular proximal catheter's dimensions. The curve was plotted as the ratio between the total hole area at each inflow position and the internal diameter 15 of a proximal catheter having an internal diameter 15 of 1.2 millimeters. The curve can be expressed mathematically by the equation F(X)=0.0699*exp[0.216*(X−8)] where F(X) is the y-coordinate of the graph at FIG. 7, X is the x-coordinate of the graph at FIG. 7, and exp is the exponent “e” (approximately 2.71828). The sixteenth inflow position of the curve is located at the most distal end of the catheter, i.e., the end furthest from the draining end of the catheter. The curve has been normalized for a sixteen inflow position catheter. FIG. 8 provides in tabular form the measurements illustrated by the curve in FIG. 7.

To design a proximal catheter by utilizing the curve, a catheter designer must first define the catheter's inner diameter 15. The inner diameter 15 of many proximal catheters is 1.2 millimeters. After selecting an inner diameter, a catheter designer intent on making a twelve inflow position catheter, for example, would merely apply the value at curve inflow position sixteen to calculate the total hole area for his or her twelfth inflow position. The designer would then apply the value at curve inflow position fifteen to calculate the total hole area at his or her eleventh inflow position. In like manner, the designer can calculate the remaining total hole areas. The designer would then select the number of inlet holes 9, 10, 11, 12, 13 desired at each inflow position 6, 7, 8 and divide each calculated total hole area by that number. Because the curve defines the total hole area at the various inflow positions, any number of inlet holes at any one inflow position may be selected. The result of this calculation for each inflow position will be the inlet hole area 16, 17, 18 for each inlet hole at each inflow position.

The present invention is intended to include all variations in the distribution of total hole areas along the longitudinal axis so long as the inlet holes 9, 11, 13 at each inflow position 6, 7, 8 progressively decrease in cross-sectional area as the inflow positions 6, 7, 8 approach the proximal end 2 of the catheter 1. The means in the exemplary embodiment for making this progressively decreasing distribution of total hole areas is but one embodiment of the present invention.

The invention is not limited by any particular shape or shapes of the inlet holes 9, 10, 11, 12, 13. It is also intended that all changes in the total hole areas resulting from altered entrance conditions of the inlet holes, such as an angled entrance or slits in the wall 4 of the catheter 1 adjacent to and in communion with the inlet hole, are within the scope of this invention.

One skilled in the art will appreciate further features and advantages of the invention based on the above-described embodiments. Accordingly the invention is not to be limited by any particular embodiments shown or described.

What is claimed is:

1. A catheter comprising an elongated tube having a series of inlet holes that progressively decrease in cross-sectional area from the most proximal inlet hole to the most distal inlet hole.

2. A catheter as defined by claim 1 wherein said decrease is essentially linear.

3. A catheter as defined by claim 1 wherein said decrease is essentially curvilinear.

4. A catheter comprising:
a) an elongated tube having a proximal end and a distal end,
b) said tube having an annular wall defining a central passageway,
c) said wall having four inflow positions along its longitudinal axis, further having one or more inlet holes at each inflow position,
d) wherein the total hole area at each successive inflow position decreases, said decrease first occurring at the inflow position immediately following the most proximal inflow position.

5. A catheter as defined by claim 4, wherein the total hole area at each successive inflow position linearly decreases.

6. A catheter as defined by claim 4, wherein the total hole area at each successive inflow position curvilinearly decreases.

7. A catheter as defined by claim 4, wherein the inner diameter of said passageway is constant.

8. A catheter as defined by claim 7, wherein the distribution of the total hole areas decreases in substantial conformance with a curved line as shown in FIG. 7.

9. A catheter as defined by claim 7, wherein the distribution of the total hole areas can be approximated by the curve created by the equation F(X)=0.0699*exp[0.216*(X−8)].
